(19) United States
(12) Patent Application Publication
(10) Pub. No.: US 2013/0330417 A1
(43) Pub. Date: Dec. 12, 2013

(54) NANOCELLULOSE FOAM CONTAINING ACTIVE INGREDIENTS

(71) Applicant: U.S. Army Research Laboratory ATTN: RDRL-LOC-I, Adelphi, MD (US)

(72) Inventors: Hong Dong, Perry Hall, MD (US); James F. Snyder, Havre de Grace, MD (US)

(21) Appl. No.: 13/912,743

(22) Filed: Jun. 7, 2013

Related U.S. Application Data
(60) Provisional application No. 61/657,259, filed on Jun. 8, 2012.

(51) Int. Cl. A61L 15/44

(52) U.S. Cl. CPC A61L 15/44
USPC 424/618; 424/646; 514/17.2; 514/54; 514/55; 514/1.1; 536/101; 530/356; 530/395; 428/338

(57) ABSTRACT

Nanocellulose foams containing at least one active ingredient and methods of preparing such nanocellulose foams containing one or more active ingredients are provided herein. In some embodiments, a method for preparing nanocellulose foam containing active ingredients may include forming a liquid mixture of nanocellulose, wherein the nanocellulose is at least one of dispersed, suspended or gelled in the liquid mixture; drying the liquid mixture of nanocellulose to form a nanocellulose foam; and mixing at least one active ingredient into at least one of the liquid mixture of nanocellulose or the nanocellulose foam. In some embodiments, a nanocellulose structure may include a nanocellulose foam comprising at least one of a carboxylate group, a hydroxyl group, or a sulfate group bonded to an active ingredient. In some embodiments, the nanocellulose structures are enhanced or crosslinked with metal cations.

### CROSS-REFERENCE TO RELATED APPLICATIONS

This application claims benefit of U.S. Patent Application Ser. No. 61/657,259, filed Jun. 8, 2012, entitled, “Nanofibrillated cellulose foam containing one or more active ingredients for wound dressing, catalysis, active filtration, and/or other applications,” which is herein incorporated by reference in its entirety.

### GOVERNMENT INTEREST

Governmental Interest—The invention described herein may be manufactured, used and licensed by or for the U.S. Government.

### FIELD OF INVENTION

Embodiments of the present invention generally relate to nanocellulose and, more particularly, to methods of preparing nanocellulose foam containing one or more active ingredients.

### BACKGROUND OF THE INVENTION

Wound dressings may be comprised of films, gels, hydrocolloids and foams. Foam wound dressings may include polyurethane foams, foams of cellulose derivatives and bacterial foams and gels. The inventors have deduced that incorporating one or more active ingredients, such as antibacterial agents and antimicrobial agents, into nanocellulose foams, also referred to as cellulose nanofibril foams, should help promote wound healing.

Therefore, the inventors have provided improved nanocellulose foams containing one or more active ingredients and methods of preparing such nanocellulose foams containing one or more active ingredients.

### BRIEF SUMMARY OF THE INVENTION

Embodiments of the present invention relate to methods of preparing nanocellulose foam containing one or more active ingredients. In some embodiments, a method of forming a nanocellulose structure may include forming a liquid mixture of nanocellulose, wherein the nanocellulose is dispersed, suspended and/or gelled in the liquid mixture; drying the liquid mixture of nanocellulose to form a nanocellulose foam; and mixing one or more active ingredients into at least one of the liquid mixture of nanocellulose or the nanocellulose foam.

In some embodiments, a nanocellulose structure may include a nanocellulose foam comprising at least one of a carboxylate group, a hydroxyl group, or a sulfate group bonded to an active ingredient.

Other and further embodiments of the invention are described in more detail below.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a flow diagram of a method of preparing nanocellulose foam containing one or more active ingredients in accordance with some embodiments of the present invention.

FIGS. 2A-2B depicts an illustrative view of a method of preparing nanocellulose foam containing one or more active ingredients in accordance with some embodiments of the present invention.

FIGS. 3A-3B depict a scanning electron micrograph of nanocellulose foam with silver nanoparticles in accordance with some embodiments of the present invention.

FIG. 4 depicts FESEM images illustrating the porous network structures of nanocellulose hydrogels.

FIGS. 5a through 5d depict the results of zone of inhibition antimicrobial tests of the nanocellulose hydrogel (a and c) and nanocellulose-Ag hydrogel (b and d) against (a-b) Escherichia coli and (c-d) Staphylococcus aureus.

### DETAILED DESCRIPTION OF THE INVENTION

Embodiments of the present invention include nanocellulose foams containing one or more active ingredients as well as methods of preparing such nanocellulose foams containing one or more active ingredients. Nanocellulose foams in accordance with embodiments of the present invention may advantageously have high surface area, porosity, and absorption and adsorption properties, as well, as biocompatibility and flexible mechanical properties.

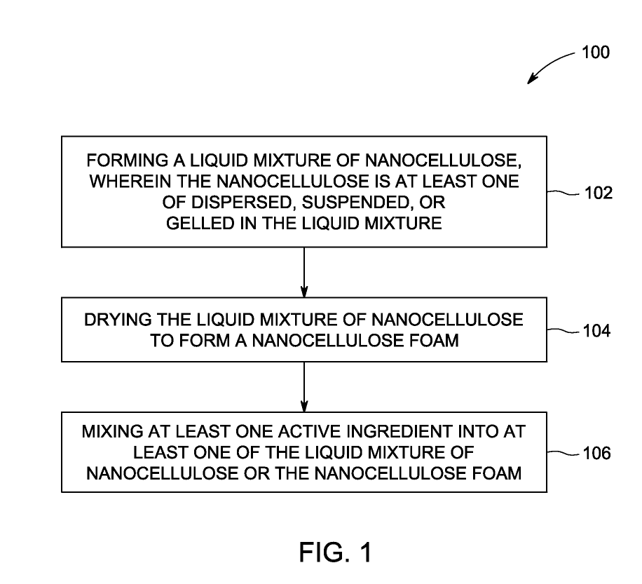

FIG. 1 depicts a flow diagram of a method 100 of preparing a nanocellulose foam containing one or more active ingredients in accordance with some embodiments of the present invention. The method 100 starts at 102 by forming a liquid mixture of nanocellulose by at least one of dispersing, suspending or gelling the nanocellulose in a liquid mixture. Nanocellulose refers to cellulosic fibrils or crystals or whiskers having a diameter of less than 1 micron, preferably less than 100 nm. The length of the nanocellulose may vary from about 10 nm to about 10 microns. The mixture of nanocellulose is formed through mechanical or chemical treatment of a cellulose containing material. In some embodiments the cellulose containing material is oxidized using 2,2,6,6-tetramethylpiperidine-1-oxyl radical (“TEMPO”). In some embodiments, acid hydrolysis, for example sulfuric acid hydrolysis, is used to produce the nanocellulose mixture. In some embodiments, the mechanical treatment is imparted by a mechanical homogenization process with or without enzymatic fractionation. In some embodiments, the cellulose containing material is one or more of wood pulp fibers, plant fibers, tunicate, algae, or ramie. Controlling the concentration of cellulose containing material in the mixture advantageously controls properties of the nanocellulose foam, such as porosity, absorption capacity, flexibility, and active ingredient release rate.

The nanocellulose produced by TEMPO oxidation is surface functionalized with carboxylate groups. The nanocellulose produced by sulfuric acid hydrolysis is surface functionalized with sulfate groups. The carboxylate groups or sulfate groups or hydroxyl groups of cellulose advantageously allow for the incorporation of a variety of active ingredients to provide a variety of functionalities, as discussed below.

At 104, the liquid mixture of nanocellulose is dried to form a nanocellulose foam. In some embodiments, the liquid mixture of nanocellulose is dried using a freeze drying process. For example, in some embodiments, the liquid mixture is frozen in an ethanol/dry ice bath then freeze dried at a pressure of 0.1 mbar. The freeze dried nanocellulose foam has an average pore size diameter of about 1 μm to about 100 μm. The pore sizes may vary from one side of the foam to another side of the foam (e.g., opposing sides). For example, a foam may be formed to have an average pore size of about 50 μm on one side and about 10 μm on another side. Alternatively, the mixture of nanocellulose can be dried using one of a super-critical carbon dioxide (CO₂) drying process or a liquid carbon dioxide (CO₂) drying process. The nanocellulose foam prepared by super-critical or liquid carbon dioxide drying comprises a pore size in the sub-micron range and a high surface area of about 200 m²/g to about 400 m²/g.

At 106, one or more active ingredients may be added to the nanocellulose. The active ingredient may be mixed into at least one of the liquid mixture of nanocellulose prior to drying 104 (discussed above) or into the nanocellulose foam after drying at 104. As used herein, an active ingredient is any chemical element, compound or other substance that can be coupled to the nanocellulose to provide additional activity that the bulk nanocellulose does not normally provide, for example pharmaceutical activity or antimicrobial activity. Some suggested active ingredients are described below in detail. In some embodiments, the active ingredient is coupled to the nanocellulose by a physical interaction, such as adhesion, or by a chemical interaction, such as covalent bonding, ionic bonding, or hydrogen bonding, or by a self-assembly process or a by vapor deposition process, or by a layer by layer process.

In some embodiments, additional materials, such as binders, proteins, surfactants, preservatives, fillers or colorants, may be added to the nanocellulose foam. Such materials can be added to the liquid mixture of nanocellulose prior to drying or to the dried nanocellulose foam. These materials can be coupled to the nanocellulose by physical or chemical interaction.

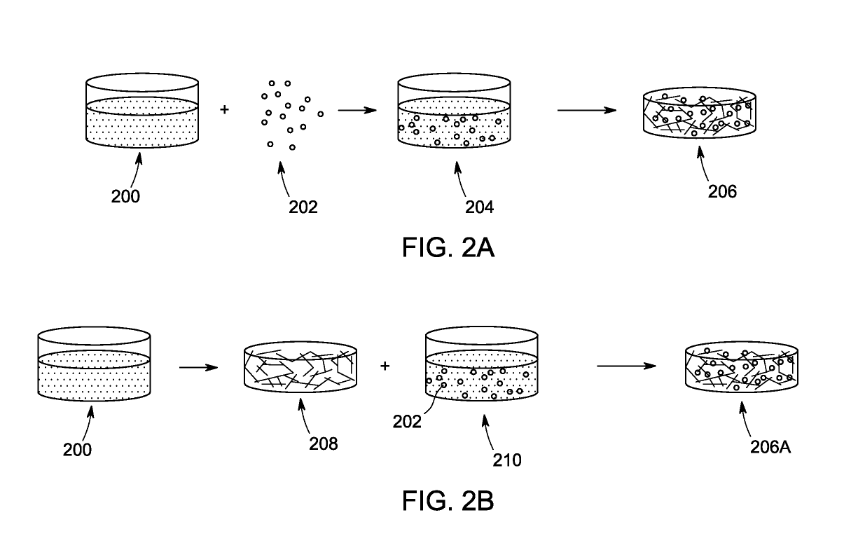

In some embodiments, as depicted in FIG. 2A, the active ingredient 202 is mixed into the liquid mixture of nanocellulose 200 to form a liquid mixture of functionalized nanocellulose 204. The liquid mixture of functionalized nanocellulose 204 is freeze-dried to form functionalized nanocellulose foam 206. The liquid mixture of functionalized nanocellulose can also be solvent-exchanged into an organic solvent, and then exposed to supercritical CO₂ or liquid CO₂ or freeze-dried to form a functionalized nanocellulose foam.

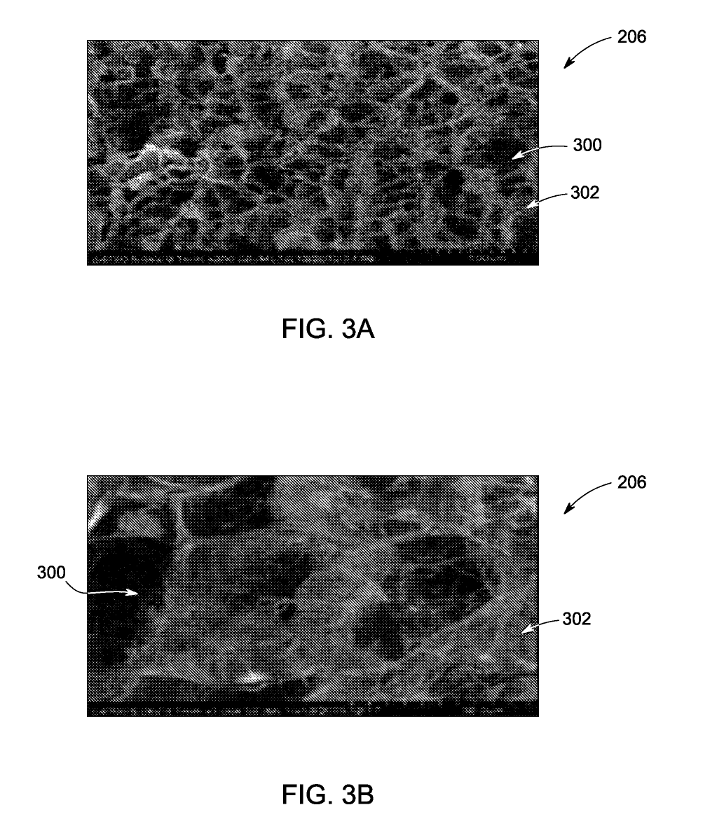

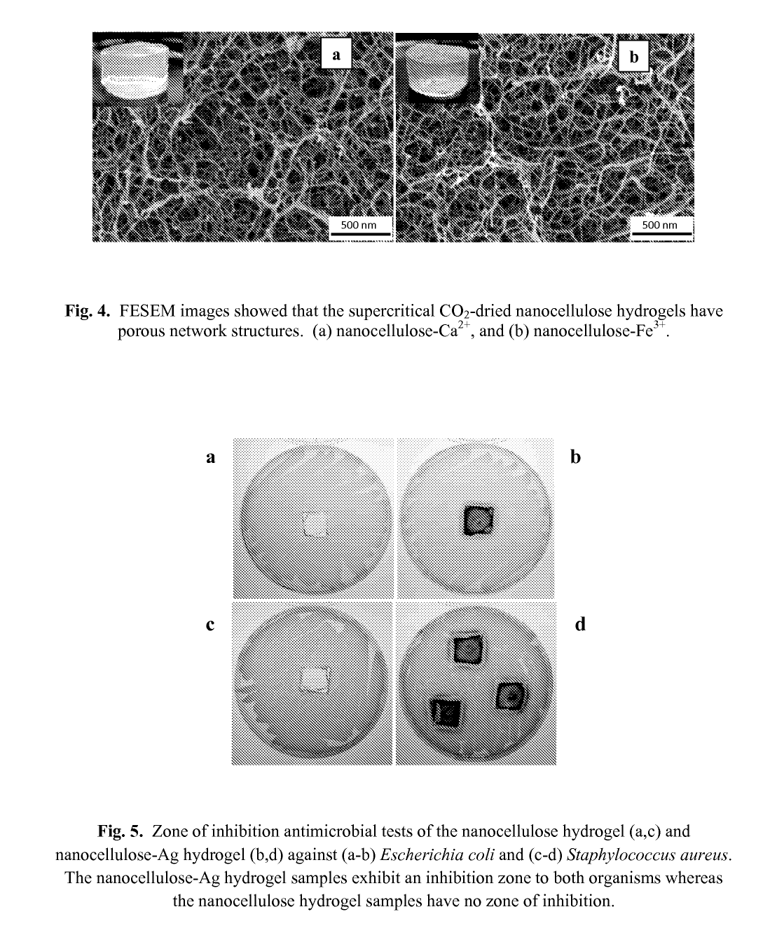

In some embodiments, the structure of nanocellulose foam is enhanced by hydrogelation of nanocellulose dispersion with cations before drying process. A few examples of these cations include, but are not limited to, Ca²⁺, Zn²⁺, Cu²⁺, Al³⁺ and Fe³⁺, among which Ca²⁺ and Fe³⁺ are biocompatible. Nanocellulose hydrogels are produced by addition of a metal salt solution to the top of nanocellulose aqueous dispersion. The moduli of thus formed hydrogels correlate well with binding strength of cations with surface carboxylate groups on nanocellulose, as provided in Table 1. FIG. 4 shows interconnected porous networks after supercritical CO₂ drying of cation-induced hydrogels. which were prepared using a method described in example 3

To include the active ingredients in cation-induced hydrogels, active ingredients can be either added to the liquid dispersion prior to hydrogelation or added to hydrogels after gel formation. For example, proteins that promote wound healing are chemically attached or physically absorbed to the surface of cation-induced hydrogels.

In some embodiments, nanocellulose gels can be functionalized with chitosan. In one example, nanocellulose beads with chitosan are generated by dropping nanocellulose dispersion into CaCl₂ or other aqueous salt solution, followed by hardening and rinsing with water. Then the nanocellulose beads were incubated with chitosan. In another example, nanocellulose dispersion was dropped into chitosan/CaCl₂ or other aqueous salt solution to form nanocellulose/chitosan beads.

In some embodiments, the liquid mixture of nanocellulose can be functionalized with silver (Ag) to form a hydrogel. For example, in some embodiments, the hydrogel is generated by adding silver nitrate (AgNO₃) to the liquid mixture of nanocellulose. In an exemplary embodiment, a sufficient amount of silver nitrate (AgNO₃) is added to the liquid mixture of nanocellulose to ensure complete saturation of carboxylate groups with silver ions. The addition of silver nitrate (AgNO₃) results in the gelation of the liquid mixture of nanocellulose. The hydrogel is allowed to sit for a desired period of time in order to promote the slow reduction from silver ions (Ag⁺) to silver (Ag) nanoparticles. The hydrogel may be immersed in water to rinse off any unattached silver (Ag) species.

In some embodiments, to form an aerogel, silver nitrate (AgNO₃) is introduced to the liquid mixture of nanocellulose in quantities to remain below the gelation threshold. The functionalized liquid mixture of nanocellulose is then degassed under vacuum to remove air bubbles and freeze dried as described above. To reduce silver ions (Ag⁺) to silver (Ag) nanoparticles, the top and bottom sides of the dried aerogels are exposed under a UV lamp for 30 minutes each. FIGS. 3A and 3B depict a scanning electron micrograph of functionalized nanocellulose foam 206 with silver nanoparticles 302, which shows the pores 300 of freeze-dried foam. The functionalized foam was prepared using a method described in example 2.

In other embodiments, as depicted in FIG. 2B, the liquid mixture of cellulose nanofibrils 200 is dried prior to adding any active ingredients, in order to form non-functionalized nanocellulose foam 208. The non-functionalized nanocellulose foam 208 is immersed in an active ingredient 202-containing solution 210 and dried to form a functionalized nanocellulose foam 206A.

For example, in some embodiments, a nanocellulose foam is prepared by adding an acid, such as hydrochloric acid (HCl), to a liquid mixture of nanocellulose resulting in the gelation of the liquid mixture. The non-functionalized nanocellulose hydrogel is removed from the hydrochloric acid (HCl) solution and washed with water several times. The hydrogel can then be dipped in a liquid solution containing an active ingredient, such as silver, and dried as described above to form a functionalized nanocellulose foam 206A.

Alternatively, for example, the nanocellulose foam is an aerogel formed by degassing the liquid mixture of nanocellulose under vacuum to remove air bubbles. The liquid mixture of nanocellulose is then freeze dried as described above. The freeze dried nanocellulose aerogel can then be loaded with an active ingredient such as silver ions or silver nanoparticles. In some embodiments, the foam can be particle or bead shapes or in sheet forms.

In some embodiments, the nanocellulose foam is used as a wound dressing and the selected active ingredient has at least one of antimicrobial properties, antiviral properties, or hemostatic properties. In some embodiments, the nanocellulose foam can have a high porosity, for example, greater than about 99%, such that upon application to the wound, the nanocellulose foam can absorb large amounts of wound fluid exudates. As the nanocellulose foam absorbs fluid, it releases the active ingredient to the wound. For example, in some embodiments, the active ingredient is at least one of a silver species, a copper species, chitosan, an antimicrobial drug, an antibiotic, a pharmaceutical, a vitamin, a mineral, or a diagnostic agent. FIG. 4 demonstrates antimicrobial properties of nanocellulose-Ag hydrogels against tested bacteria. Nanocellulose-Ag hydrogels were prepared using a method illustrated in example 1.

A variety of active ingredients can be added to the liquid mixture of nanocellulose suitable for use in a variety of industries, such as biomedical, cosmetic, and pharmaceutical. In some embodiments, the active ingredient is advantageously selected to promote a variety of properties, such as adsorption of external materials, permeability of matter or energy, conductivity, catalysis, biological activity, reactivity, electrochemical reactions, or mechanical properties.

For example, in some embodiments, the nanocellulose foam is a tissue scaffold and the active ingredient is selected to provide stability and attachment for cell growth. In such embodiments, the active ingredient is at least one of collagen, chitosan, hyaluronic acid, or proteins.

In some embodiments, the active ingredient has high adsorption or absorption properties, which can be useful in applications such as wound dressings or diapers.

In some embodiments, the active ingredient is selected to bind, trap, or filter target materials in liquid or gas phase effluent, which is useful in applications such as air purification, water sanitization or wastewater treatment.

In some embodiments, the active ingredient has a high electrical conductivity, which is useful in a variety of applications including but not limited to electronics or protection against stray current (e.g., lightning strike). In such embodiments, the active ingredient is, for example, a metal species such as copper, silver, gold, or platinum, or an electrically conducting polymer, such as polypyrrole, polyaniline, or poly(3,4-ethylenedioxythiophene). In some embodiments, the active ingredient has high electrical resistivity, which is useful in a variety of applications including but not limited to electrical shielding or electronics.

In some embodiments, the active ingredient has either thermally conductive properties, such as silver, copper or aluminum oxide, or has thermal insulation properties, such as rubber, silica, or polyethylene. Such properties are useful in a variety of applications including but not limited to insulation or thermoelectrics.

In some embodiments, the active ingredient provides acoustic dampening properties which are useful in a variety of applications including but not limited to sound insulation in buildings.

In some embodiments, the active ingredient is a non-linear optical material, such as lead pthalocyanine and related derivatives.

In some embodiments, the active ingredient interacts with electromagnetic waves. In some embodiments, the active ingredient reflects energy in the form of electromagnetic waves, sound, or heat so as to provide a waveguide through the nanostructure, which is useful in a variety of applications.

In some embodiments, the active ingredient can store energy, which is useful in a variety of applications including but not limited to electrochemical batteries or capacitors. In some embodiments, the active ingredient can undergo oxidative or reductive changes to store ionic or electric charge. In such embodiments, the active ingredient is at least one of a redox-active polymer, such as polyaniline or polypyrrole, a transition metal, such as lithium, cobalt oxide, lithium manganese oxide, or lithium iron phosphate, carbon, such as graphite or carbon nanotubes, silicon, tin, lithium, sodium, lead, or other electrode materials.

In some embodiments, the active ingredient has chemically active properties. In some embodiments, the active ingredient has catalytic properties. In some embodiments, the active ingredient is a gas-phase catalyst and is selected from a group consisting of a noble metal or a metal alloy catalyst. In some embodiments, the active ingredient is a liquid-phase catalyst and is selected from a group consisting of a noble metal or a metal alloy catalyst.

In some embodiments, the active ingredient reacts with chemical or biological agents to render them inert, for example, titanium oxide.

In some embodiments, the active ingredient can react with an external stimulus, such as increased temperature or an applied voltage to generate a detectable chemical, mechanical, or electrical signal, which is useful in a variety of sensor applications.

In some embodiments, the active ingredient has mechanical properties that change based on external stimuli.

In some embodiments, the active ingredient has magnetic properties, which is useful in a variety of applications including but not limited to electric generators or data recording. In such embodiments, the active ingredient is, for example, at least one of a ferrite or a rare-earth-element-based complex such as samarium-cobalt or an alloy of neodymium, iron and boron.

### EXAMPLE 1

Nanocellulose-Ag hydrogels were generated by addition of AgNO₃ aqueous solution to an aqueous dispersion of carboxylated nanocellulose followed by reduction. Typically, nanocellulose dispersion was put into a container. An equal volume of 50 mM AgNO₃ solution was added dropwise along the sidewall into the 1 wt % nanocellulose dispersion without stirring. Gelation occurred rapidly upon the addition of AgNO₃. The gel sat for five days to allow for slow reduction of Ag⁺ to Ag nanoparticles. UV reduction as an alternative method could also be used to convert Ag⁺ to Ag nanoparticles. A brown gel thus formed was removed from the AgNO₃ solution, and immersed into water several times to rinse off the unattached Ag species.

### EXAMPLE 2

A freeze-drying method was used to prepare nanocellulose-Ag aerogels. The molar amount of AgNO₃ added to the 1 wt % nanocellulose dispersion was calculated on the basis of the dried nanocellulose weight. Low quantities were desired to remain below the gelation threshold. To 40 g of nanocellulose aqueous dispersion, the calculated amount of AgNO₃ corresponding to 0.2 mmol or 0.5 mmol Ag⁺ per gram of dried nanocellulose was dissolved in 1 mL of H₂O and added dropwise under vigorous stirring. After continuously stirring for 30 min, the aqueous dispersion was degassed quickly under vacuum. 8 grams of each sample were put in a glass freeze-drying vial and immersed in an ethanol/dry ice bath. An ethanol/dry ice bath was preferred over liquid N₂ for freezing the NFC dispersion as it was found to generate fewer cracks in the aerogel structures. The frozen dispersion was then freeze-dried at a pressure of 0.1 mbar in a FreeZone freeze dry system. The drying was typically finished within 12-24 h. To reduce Ag⁺ to Ag nanoparticles, the dried aerogels were exposed under a UV lamp (λ=320-395 nm) 30 min each for the top side and the bottom side.

### EXAMPLE 3

Nanocellulose hydrogels were produced by addition of a metal salt solution to the top of aqueous dispersion of carboxylated nanocellulose. A certain weight of 1 wt % nanocellulose dispersion was put in a container. An equal weight of a 50 mM aqueous solution of metal salt, such as CaCl₂ or FeCl₃, was added dropwise along the wall of the container into the CNF dispersion without stirring. Gelation occurs upon the addition of the metal salt solution. After standing for overnight, the metal salt solution was decanted, the resulting hydrogel was soaked and rinsed with water several times to remove unbounded metal ions. For the hydrogel generated with FeCl₃, a yellow gel formed after addition of 50 mM FeCl₃. t, the gel of CNF—Fe³⁺ was rinsed with water of pH 3 before rinsing with neutral water.

The hydrogels in example 1 and 3 were dried either by freeze-drying using similar conditions as described in example 2 or by sc-CO₂ drying after solvent exchanged with acetone.

### EXAMPLE 4

1 wt % nanocellulose dispersion was pumped through a syringe into a gelling bath that contained an aqueous solution of 50 mM CaCl₂ solution. The gel beads were allowed to harden in the gelling bath for 1 hour, and then rinsed with water. The gel beads were then incubated with buffered chitosan solution for overnight.

Other details and/or embodiments may be described in a journal article titled “Hydrogel, aerogel and film of cellulose nanofibrils functionalized with silver nanoparticles” Carbohydrate Polymers 95 (2013 760-767) which is hereby incorporated by reference.

While the foregoing is directed to embodiments of the present invention, other and further embodiments of the invention may be devised without departing from the basic scope thereof.

TABLE 1
Moduli of the nanocellulose hydrogels that were crosslinked with divalent
and trivalent cations

| Metal Ions | | | | | | |
|---|---|---|---|---|---|---|
| (concentration: | | | | | | nanocellulose |
| 50 mM) | Fe³⁺ | Al³⁺ | Cu²⁺ | Zn²⁺ | Ca²⁺ | dipersion |
| logK1* (CH₃COO⁻) | 4.00 | 3.13 | 2.22 | 1.49 | 0.44 | — |
| [symbol] Mⁿ⁺ gel G′ at | 37.4 | 30.9 | 14.7 | 5.75 | 3.8 | 0.01 |
| 1 Hz (kPa) | | | | | | |

1. A method of forming a nanocellulose structure, comprising:
forming a liquid mixture of nanocellulose, wherein the nanocellulose is at least one of dispersed, suspended or gelled in the liquid mixture;
drying the liquid mixture of nanocellulose to form a nanocellulose foam; and
mixing at least one active ingredient into at least one of the liquid mixture of nanocellulose or the nanocellulose foam.

2. The method of claim 1, wherein the active ingredient comprises at least one of antimicrobial agents, antiviral agents, pharmaceutical agents, antibiotics, vitamins, minerals, or diagnostic agents.

3. The method of claim 1, wherein the active ingredient comprises at least one of collagen, chitosan, hyaluronic acid, or proteins.

4. The method of claim 1, wherein the nanocellulose structures are enhanced or crosslinked with metal cations.

5. The method of claim 1, wherein the active ingredient comprises at least one of a metal species or an electrically conducting polymer.

6. The method of claim 1, wherein the active ingredient comprises at least one of a redox-active polymer, a transition metal complex, carbon, silicon, tin, lithium, or sodium.

7. The method of claim 1, wherein the active ingredient is at least one of a gas-phase catalyst or a liquid phase catalyst.

8. The method of claim 1, further comprising adding at least one of binders, proteins, surfactants, preservatives, fillers, or colorants to at least one of the liquid mixture of nanocellulose or to the nanocellulose foam.

9. The method of claim 1, wherein the nanocellulose foam comprises open pores having a pore size of about 1 nm to about 1000 μm.

10. The method of claim 1, wherein drying the liquid mixture further comprises drying the liquid mixture by freeze drying the liquid mixture.

11. The method of claim 1, wherein drying the liquid mixture further comprises drying the liquid mixture by one of super-critical carbon dioxide drying or liquid carbon dioxide drying of the liquid mixture.

12. The method of claim 1, wherein the active ingredient is silver.

13. The method of claim 12, further comprising:
freeze drying the liquid mixture of nanocellulose to form the nanocellulose foam;
soaking the nanocellulose foam in a silver salt solution;
rinsing the nanocellulose foam in a solvent; and
drying the nanocellulose foam to remove solvent from the nanocellulose foam.

14. A nanocellulose structure, comprising:
a nanocellulose foam comprising at least one of a carboxylate group or a hydroxyl group or a sulfate group bonded to an active ingredient.

15. The nanocellulose structure of claim 134, wherein the active ingredient is chemically bonded to at least one of the carboxylate group or the hydroxyl group or the sulfate group.

16. The nanocellulose structure of claim 14, wherein the nanocellulose foam comprises open pores having a pore size of about 1 nm to about 1000 μm.

17. The nanocellulose structure of claim 14, wherein the active ingredient comprises at least one of antimicrobial agents, antiviral agents, pharmaceutical agents, antibiotics, vitamins, minerals, or diagnostic agents.

18. The nanocellulose structure of claim 14, wherein the active ingredient comprises at least one of collagen, chitosan, hyaluronic acid, or proteins.

19. The nanocellulose structure of claim 14, wherein the active ingredient comprises at least one of a metal species or an electrically conducting polymer.

20. The nanocellulose structure of claim 14, wherein the active ingredient comprises at least one of a redox-active polymer, a transition metal complex, carbon, silicon, tin, lithium, or sodium.

21. The nanocellulose structure of claim 14, wherein the active ingredient is at least one of a gas-phase catalyst or a liquid phase catalyst
